(19) United States
(12) Patent Application Publication
(10) Pub. No.: US 2011/0224678 A1
(43) Pub. Date: Sep. 15, 2011

(54) METHOD AND IMPLANTATION SYSTEM FOR IMPLANTING A CARDIOVASCULAR PROSTHESIS

(76) Inventor: SHLOMO GABBAY, Boca Raton, FL (US)

(21) Appl. No.: 12/886,087

(22) Filed: Sep. 20, 2010

Related U.S. Application Data
(63) Continuation-in-part of application No. 11/387,163, filed on Mar. 23, 2006, now abandoned.

(51) Int. Cl. A61F 2/00

(52) U.S. Cl. 606/108

(57) ABSTRACT

A method of implanting a cardiac prosthesis is disclosed. The method can include placing a cardiac prosthesis into an elongated barrel of an implanter, the barrel comprising an open discharge end. An introducer apparatus is attached onto the open discharge end of the barrel, the introducer apparatus comprising a sidewall portion that extends from a first end portion and terminates in a distal end portion that gradually tapers to cross-sectional dimension that is less than cross-sectional dimension at the first end portion. The introducer apparatus can be configured to permit movement of the barrel, including the discharge end, axially through and beyond the distal end portion of the introducer apparatus. An opening can be created in tissue that provides a path from the opening to an implantation site. The introducer apparatus is inserted into the opening and the barrel axially can be moved through the introducer apparatus to position the discharge end of the barrel near the implantation site. The prosthesis can then be discharged from the barrel at the implantation site.

### CROSS-REFERENCE TO RELATED APPLICATIONS

This application is a continuation-in-part of co-pending U.S. patent application Ser. No. 11/387,163, which was filed Mar. 23, 2006, and titled IMPLANTATION SYSTEM AND METHOD FOR LOADING AN IMPLANTER WITH A PROSTHESIS, which is incorporated herein by reference in its entirety.

### BACKGROUND

Various types of implantable cardiovascular prostheses have been developed and corresponding approaches are utilized to implant prostheses in both human and non-human patients. For example, it is known to utilize annuloplasty rings, stents other implantable cardiac prosthetic devices for helping improve functionality of a patient's heart valve. Other types of valves (e.g., venous valves) and stents can be utilized to improve circulation in veins and other blood vessels.

In severe cases of valvular defect and/or deficiency, implantable heart valve prostheses, such as natural tissue valves, mechanical valves and biomechanical valves are employed to replace a defective valve. In most cases, to surgically implant these and other cardiac prostheses into a patient's heart, the patient typically is placed on cardiopulmonary bypass during a complicated, but common, open chest and, usually, open-heart procedure. In an effort to reduce risk to the patient, minimally invasive implantation techniques for various cardiac prostheses are continually being developed and improved, including those shown and described in the above-incorporated patent applications.

### SUMMARY

The present disclosure relates generally to an implantation system and method for implanting cardiovascular prostheses.

One embodiment provides a method for implanting a cardiovascular prosthesis. The method can include placing a cardiac prosthesis into an elongated barrel of an implanter, the barrel comprising an open discharge end. An introducer apparatus is attached onto the open discharge end of the barrel, the introducer apparatus comprising a sidewall portion that extends from a first end portion and terminates in a distal end portion that gradually tapers to cross-sectional dimension that is less than cross-sectional dimension at the first end portion. The introducer apparatus can be configured to permit movement of the barrel, including the discharge end, axially through and beyond the distal end portion of the introducer apparatus. An opening can be created in tissue that provides a path from the opening to an implantation site. The introducer apparatus is inserted into the opening and the barrel axially can be moved through the introducer apparatus to position the discharge end of the barrel near the implantation site. The prosthesis can then be discharged from the barrel at the implantation site.

Another embodiment can provide an implantation system. The system can include an elongated barrel extending longitudinally from a proximal portion to terminate in an open discharge end, a body lumen extending through the barrel to provide for fluid communication through the barrel. A plunger is configured for discharging a prosthesis from the discharge end of the barrel. An introducer apparatus is attachable over the discharge end of the barrel. The introducer apparatus can include a sleeve portion that extends from a first end and a distal portion that tapers gradually from the sleeve portion and terminates in a distal end thereof. The introducer apparatus is configured to permit movement of the barrel, including the discharge end, axially through and beyond the distal end of the introducer apparatus. The implantation system can be used as part of a method for implanting the prosthesis.

### BRIEF DESCRIPTION OF THE DRAWINGS

FIG. 1 depicts an assembly view of an example embodiment of an implantation apparatus.

FIG. 2 depicts an assembled view of the implantation apparatus of FIG. 1.

FIG. 3 depicts an assembly view of another example embodiment of an implantation apparatus.

FIG. 3A depicts an alternative example embodiment of a body that can be utilized in the implantation apparatus of FIG. 3.

FIG. 4 depicts an assembled view of the implantation apparatus of FIG. 3.

FIG. 5 depicts a first portion of an example embodiment of a procedure for loading a prosthesis into an implantation apparatus.

FIG. 6 depicts a second portion of the procedure of FIG. 5 for loading the prosthesis into the implantation apparatus.

FIG. 7 depicts a first portion of an example embodiment of a procedure for loading a prosthesis into an implanter using a loading system.

FIG. 7A depicts a front view of the pusher member in a first condition taken along line 7A-7A in FIG. 7.

FIG. 8 depicts a second portion of the procedure of FIG. 7.

FIG. 8A depicts a sectional view taken along line 8A-8A in FIG. 8, illustrating the pusher member in a second condition.

FIG. 9 depicts an example embodiment of an implantation apparatus being urged through an introducer apparatus.

FIG. 10 depicts an example embodiment of an introducer apparatus being inserted at an aorta of a heart.

FIG. 11 depicts an example embodiment of a valve being implanted at an aortic position.

FIG. 12 depicts an example embodiment of an introducer apparatus being inserted near an apex of a heart.

FIG. 13 depicts an example embodiment of a valve being implanted at the pulmonic position.

FIG. 14 depicts another example embodiment of an introducer apparatus.

FIG. 15 depicts an example embodiment of an implantation apparatus being urged into the introducer apparatus of FIG. 14.

FIG. 16 depicts an example embodiment of an implantation apparatus being urged through the introducer apparatus of FIG. 14.

FIG. 17 depicts a first portion of an example embodiment of a procedure for implanting a prosthesis.

FIG. 18 depicts a second portion of an example embodiment of a procedure for implanting a prosthesis.

### DETAILED DESCRIPTION

In the area of minimally invasive cardiovascular surgery, several types of prostheses, including heart valves, venous valves, stents, annuloplasty rings and other apparatuses, can be compressed to a smaller diameter to facilitate their positioning to a desired implantation site (e.g., within a patient's heart). For instance, many such devices may have a substantially C-shaped or substantially annular cross-sectional configuration when in an expanded state, as intended for replacing or augmenting operation of anatomical features, such as a heart valve. Some of the prostheses intended for minimally invasive surgical implantation include spikes, barbs or other protrusions that extend outwardly from the prosthesis. Accordingly, when handling the prosthesis, traditional sterile gloves can rip or be punctured by the spikes or barbs. This disclosure provides an implantation system (or apparatus) and method to facilitate the implantation of a prosthesis.

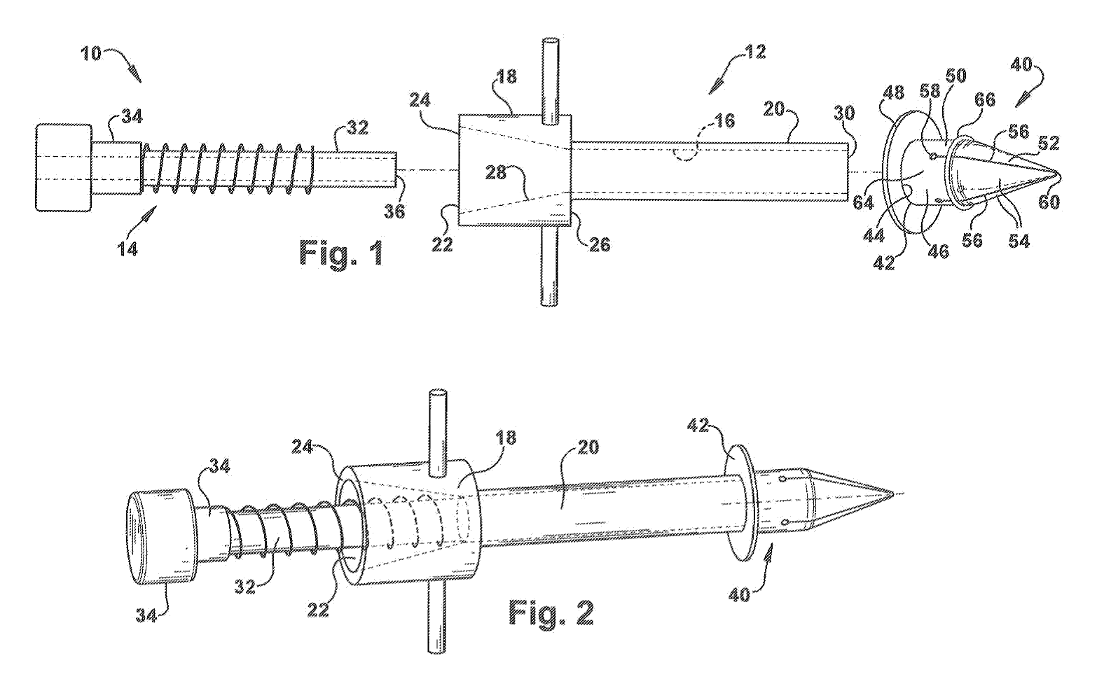

FIGS. 1 and 2 depict an example of an implantation system (also referred to herein as an implanter or implantation apparatus) 10. The implanter 10 includes an elongated body portion 12 and a plunger 14 that is moveable within a lumen 16 extending at least partially through the body portion.

The body portion 12 includes a loading portion 18 and a barrel 20 that extends longitudinally from the loading portion. The loading portion 18 is configured to facilitate loading a prosthesis into the barrel 20. The loading portion 18 includes an opening 22 at a first end 24 that is spaced apart from a second end 26 of the loading portion by a substantially conical interior sidewall portion 28. The diameter at the opening 22 is greater than the inner diameter adjacent the second end 26 of the loading portion 18. That is, the interior sidewall 28 tapers from the larger diameter at the opening 22 to the smaller diameter adjacent the second end 26. The particular dimension of the interior sidewall 18 can be configured according to the starting (or expanded) size of prosthesis and the desired ending (or reduced cross-sectional) size of the prosthesis. As described herein, the desired ending size will be defined by the inner diameter of the barrel 20. Accordingly, adjacent the second end 26, the diameter of the interior sidewall 28 is commensurate with the inner diameter of the barrel 20, thereby providing a substantially smooth transition between such parts.

In the example of FIGS. 1 and 2, the interior sidewall portion 28 is illustrated as having a conical frustum (or frusto-conical) cross sectional configuration that extends between the opening 22 and the second end 26 of the loading portion 18. For example, the interior sidewall portion 28 may be configured with an angle that is less than approximately 45 degrees, such as in a range from about 10 degrees to about 20 degrees, relative to a central longitudinal axis extending through lumen 16. It is to be understood that the shape of the loading portion 18 is not limited to the shape of a conical frustum. For example, other shapes or combinations of shapes, including one or more curved portions, can be used to provide the tapering interior sidewall 28 of the loading portion 18.

The barrel 20 extends a desired distance from the loading portion to terminate at an opening at a distal end 30, which distance can vary according to the type of prosthesis or location at which the prosthesis is to be implanted. In the example of FIGS. 1 and 2, the lumen 16 within the barrel 20 has a diameter that is substantially fixed along its length, generally corresponding to the diameter of the sidewall 28 adjacent the end. A fixed lumen diameter in the barrel 20 is not required, however. For example, the diameter of the lumen 16 in the barrel 20 might be slightly greater (e.g., from about 1 mm to about 2 mm greater) than the diameter of the sidewall at the interior juncture of the lumen between the loading portion 18 and the barrel 20.

The loading portion 18 and the barrel 20 can be formed as a monolithic structure to define the body portion 12. By monolithic structure, it is meant that the loading portion 18 and the barrel 20 are integrally formed as a single piece; although, it does not require that the structure include only one type of material or that the portions are of the same material. The body portion 12 can be formed of one or more materials. Those skilled in the art will understand and appreciate various manufacturing techniques that can be employed to make the body portion 12, including injection molding, stamping, casting, extrusion, machining, to name a few, or any combination thereof. The body portion 12 is not limited to any of method of manufacture, however.

As mentioned above, the plunger 14 is configured to traverse the lumen 16. The plunger 14 can be implemented as including an elongated rod 32 that extends from a proximal end portion 34 and terminates in a contact end 36. The contact end 36 is dimensioned and configured to traverse the lumen 16 within the barrel 20 as well as to engage a proximal end of a prosthesis that has been inserted therein for implantation. Accordingly, the elongated plunger rod 32 can be at least as long as the barrel 20 to facilitate discharging the prosthesis completely from the end 30 of the barrel. While a spring is shown around the rod 32 of the plunger, such spring or other biasing mechanism could, alternatively or additionally be disposed within the barrel 20 of the body portion 12.

The implanter 10 can also include an introducer apparatus 40 that can be attached adjacent the end 30 of the barrel 20. The introducer apparatus 40 includes a flange 42. While the flange 42 is illustrated as a complete annular flange, those skilled in the art will understand and appreciate that the flange 42 can be implemented in a variety of shapes (e.g., rectangular hexagonal, etc.) and that flange 42 need not extend in a complete annular structure. For example, the flange 42 can be implemented as substantially co-planner tabs, a rim, as well as a curved or c-shaped flange portion or as a thicker sidewall portion of the apparatus 40.

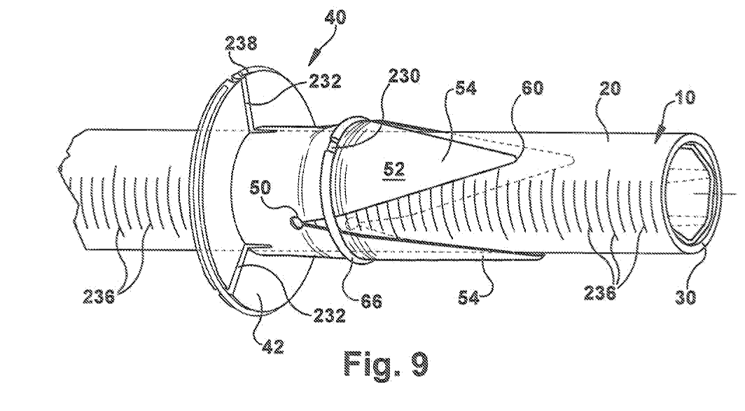

In the example of FIGS. 1 and 2, the flange 42 includes an inner periphery 44 that defines an opening into an associated sleeve 46. While the inner periphery is 44 generally circular in the examples of FIGS. 1 and 2, it can have other shapes, generally corresponding to the shape of the barrel 20. The flange 42 also includes an outer periphery 48 spaced apart from the inner periphery 44 by an associated intermediate portion thereof. The sleeve 46 has a sidewall 50 that extends longitudinally from the flange 42 and terminates in a distal end portion 52. A central axis extends through a center of the sleeve 46 and through the opening defined by the inner periphery 44 of the flange 42. An adjustable opening is operatively associated with the distal end portion 52. The adjustable opening includes means movable from at least a closed condition (FIG. 1) to an open condition (FIG. 9) to permit substantially free movement of an article, such as an implanter barrel or trocar, axially through a passage defined by the sidewall 50.

By way of example, the adjustable opening in the distal end portion 52 can include two or more jaw members 54 that are movable in a generally radially direction relative to the central axis between the open and closed conditions. In the example of FIGS. 1 and 2, the distal end portion 52 includes a plurality of three jaw members 54 positioned in closed condition. Longitudinally extending side edges of adjacent jaw members 54 define longitudinally extending slits 56. The longitudinally extending slits 56 extend through the sidewall 50 from a first position 58, which is located proximal to the flange 42, to intersect at an opposed end 60 of the distal end portion 52 to separate the jaw members 54. An aperture further may extend through the sidewall 50 of the sleeve at the first position 58 of each of the respective slits 56. The respective apertures operate as hinges to facilitate the generally radial movement of the respective jaw members 54 from the closed condition to the open condition. Other structure, such as hinges, can also be employed to provide for moveability of the jaw members 54. Additionally, one of the slits 56 might extend completely through the sidewall 50 as well as through the flange 42 to accommodate differently sized elongate members within the passage. Thus, the sidewall 50 can be generally cylindrical (such as shown in FIGS. 1 and 2, or the sidewall can be generally C-shaped.

In the example of FIG. 1, the sleeve 46 can include an intermediate portion 64 located between the flange 42 and the distal end portion 52. The intermediate portion 64 of the sleeve 46 can have a generally right circular cylindrical cross section that extends a predetermined length of from the flange substantially coaxial with the axis. The inner sidewall portion of the intermediate portion 64 is dimensioned and configured to fit snuggly over the distal end 30 of the barrel 20. The remainder of the sleeve 46, corresponding to the distal end portion 52, has a generally conical shape when in the closed condition depicted in FIG. 1. As an example, the slits 56 extend from the end 60 of the distal end portion 52 through the sidewall 50 to the position 58 which can be located between the respective ends of the intermediate portion 64 of the sleeve 46. Those skilled in the art will appreciate that the intermediate portion 64 and distal end portion 52 may have other shapes than as shown and described herein.

The introducer apparatus 40 can be formed of a variety of materials including metals, alloys polymers and/or composites, although it should be made of a material that is considered biocompatible or that can be made sufficiently biocompatible for at least temporary insertion into a desired tissue of a patient. Additionally, the flange 42 and sleeve 46 can be formed of the same or different materials. When formed of the same material, such as a plastic or thermoplastic material, the entire implanter 10 can be injection molded from a common material. Different materials can also be utilized for the various parts of the implanter 10.

The sidewall 50 of the sleeve 46 has a thickness that can be selected according to the material utilized for the sleeve to enable desired movement of the distal end portion to between the opened and closed conditions thereof. For instance, the sleeve 46, or at least the jaw members 54 thereof, can be formed of a flexible or pliant material to facilitate movement of the jaw members from the closed to open condition, such as upon insertion of an elongate member therethrough. The materials utilized might also be an in elastically deformable material so that jaw members 54 may remain in a substantially open condition after removal of the elongate device. No amount of resilience or memory of the jaw members or memory of the material is required for implementing the introducer apparatus 40.

Additionally, the introducer apparatus 40 can include means, such as an annular structure (e.g., a flexible ring or rubber band) 66, to inhibit movement of the distal end portion from the closed condition to the open condition. The annular structure 66 also inhibits relative movement of the elongate member through the introducer apparatus 40. In FIGS. 1 and 2, the structure 66 is depicted as a ring mounted around an exterior of the distal end portion 52 of the sidewall 50 of the introducer apparatus 40. Those skilled in the art will understand and appreciate other shapes (C-shaped or U-shaped) and configurations of structures that can be applied to the sidewall 50 to implement the functions described herein as being performed by the structure 66.

In the example of FIGS. 1 and 2, the annular structure 66 applies a radially inward force to help hold the jaw members 54 in the substantially closed position. As a result, the end 30 of the barrel 20 generally is only insertable until the end 30 engages an interior surface of the distal end portion. By inhibiting insertion of the elongate member through the apparatus 40, the annular structure 66 facilitates insertion of the combination of the elongate member and introducer apparatus 40 into an anatomical structure of a patient. The ring structure 66 also operates to maintain the distal end portion 52 and jaw members 54 in a generally conical arrangement as it is urged into an anatomical structure for implantation.

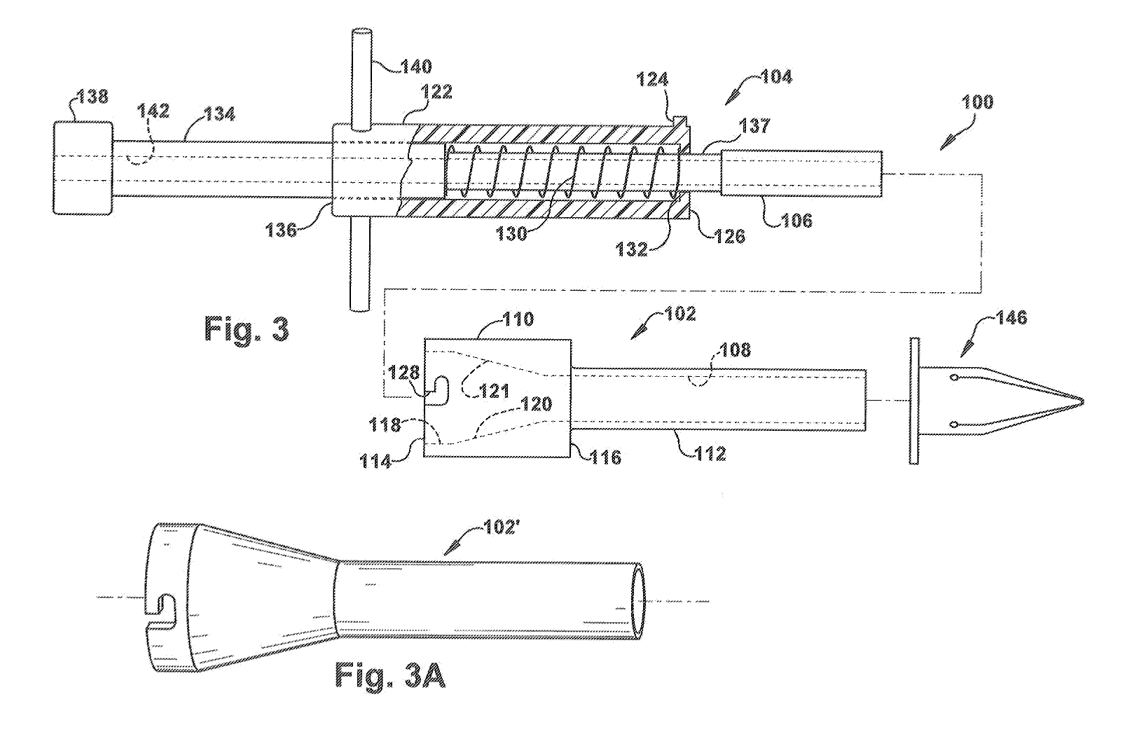

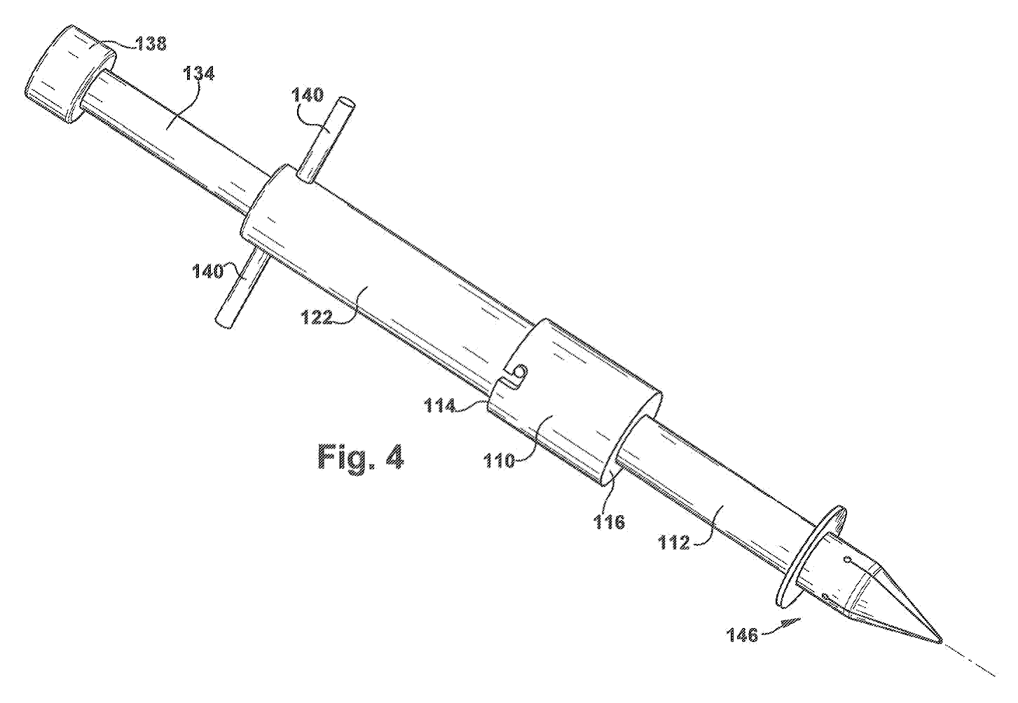

FIGS. 3 and 4 depict another example of an implantation system 100 that can be implemented. The implantation system 100 includes an elongated body portion 102 and a plunger assembly 104. The plunger assembly 104 includes a plunger member 106 that is movable axially within a lumen 108 that extends through a body portion 102. Similar to the example of FIG. 1, the body portion 102 includes a loading portion 110 and an elongated barrel 112 that extends axially from the loading portion. Loading portion 110 includes an opening at a first end 114 that is spaced apart from a second end 116 of the loading portion by a corresponding interior sidewall 120. The interior sidewall 120 of the loading portion 110 is dimensioned to facilitate insertion and loading of a prosthesis into the lumen 108 of the barrel 112, such as by causing the prosthesis to compress to a reduced diameter relative to its expanded configuration as it moves axially through the loading portion. The interior sidewall 120 of the loading portion 110 is in fluid communication with the interior of the barrel 112 to facilitate axial insertion of the prosthesis through the loading portion and into the barrel.

In the example of FIG. 3, the interior sidewall 120 of the loading portion 110 has tapering cross-sectional diameter. A first portion 118 adjacent the opening at 114 can have a substantially circular cylindrical configuration and a second portion 121 between the first portion 118 and the barrel 108 has a substantially frusto-conical configuration. The size of the loading portion and barrel should be commensurate with the expanded and reduced cross-sectional size of the prosthesis to be implanted. The cross-sectional dimension of the first portion 118 should be substantially equal to or slightly greater than a prosthesis in its expanded condition to facilitate its insertion into the first portion. Alternatively, for a large diameter prosthesis, it may be appropriate to reduce the size partially manually to enable insertion into the first portion of the loading portion. The lumen 108 and exterior surface of the barrel 112 can have a substantially constant cross-sectional diameter along its length. The diameter of the lumen 108 might also vary along its length.

The plunger assembly 104 is configured to connect with the loading portion 110 of the body 102. The plunger assembly 104 can be releasably attached to the body 102, such as by a mechanical cooperation between different parts that operates to hold plunger assembly together to with the body. In the example of FIGS. 3 and 4, the plunger assembly 104 includes a housing 122 that includes a protection or tab 124 located adjacent a distal end 126. The distal end 126 of the housing 122 is dimensioned and configured for insertion within the first portion 118 of the loading portion 110. The projection or tab 124 can be inserted into a notch 128 formed through the side wall of the loading portion 110. For example, the notch 128 can be formed of a substantially L-shaped aperture that extends through and from the first end 114 of the loading portion 110 for a distance that is less than the axial distance of the first portion 118 and then extend circumferentially another distance, which can approximate or be different from the first distance. The projecting member 124 and the distal end of the plunger assembly 104 thus can be inserted within the first portion 118 of the loading portion 110 such that the projection aligns with and into the notch 128. The plunger assembly 104 can be rotated relative to the body 102 about the central axis, such that the projection extends down the circumferential extending portion of the notch 128. The relative rotation effectively locks the plunger assembly 104 with the body 102. In this configuration, the plunger 106 that extends from the distal end 126 of the plunger assembly 104 may also traverse the lumen 108 through the body 102. Those skilled in the art will understand and appreciate various other means than can be implemented to releasably mount the plunger assembly 104 with the body 102. For example, a friction fitting, one or more clamps, one or more latches, pins or set screws, or a combination of these or other means can be utilized for attaching plunger assembly 104 with the body 102.

A spring (or other biasing means) 130 can provide for some resistance to the axial movement of the plunger 106 relative to the housing 122. For example, the spring 130 can include a distal end that engages or is attached within the housing 122, such as at a shoulder portion 132 that is located within the housing adjacent the end 126. A proximal end of the spring 130 can engage a rod 134 that extends from a proximal end 136 of the housing and terminates in a knob 138. For example, the spring 130 can circumscribe a portion of an elongated member 137 that interconnects the rod and the plunger member 126; namely, the portion of the elongated member that is located within the housing 122. The amount of tension provided by the spring 130 can be varied to provide a desired ergonomic feel for the user.

The rod 134 is dimensioned and configured for axial movement within the housing 122. The rod 134, the elongated member 137 and the plunger member 126 can thus be formed of one or more structures that are connected together to provide the elongated plunger mechanism, such as shown in FIG. 3. To effect movement of the plunger member 106 relative to the housing 122, such as through the body lumen 108 when the plunger assembly and the body are attached together, the knob 138 and associated rod 134 can be urged axially into to the housing 122. The advancement of the plunger member 106 can be implemented, for example, by a user gripping a pair of radially extending handles with his/her index and pointer fingers while concurrently pushing the knob axially with his/her thumb (similar to a syringe). Additionally, while a pair of arms 140 are depicted as extending radially from the housing 122, such design is not restricted to any number of arms, as there can be any number of arms or such arms may be omitted. Those skilled in the art will appreciate other arrangements and mechanisms (e.g., mechanical, electrical or a combination of mechanical and electrical) that can be utilized for advancing the plunger 106 through the lumen 108. Also depicted in the example of FIG. 3, an aperture or lumen extends longitudinally through the plunger assembly 104. The lumen 142 permits the movement of other articles (e.g., an elongated catheter, sutures, trocar or a combination of articles) through the assembled implanter system 100.

The implanter 100 can also include an introducer apparatus 146, which may be the same as described with respect to FIGS. 1 and 2. Additional information about the introducer apparatus 146 thus may be had by reference back to FIGS. 1 and 2 and the relevant description.

FIG. 3A depicts an example of an alternative configuration of the body portion 102′ from that shown in FIG. 3. In the configuration of FIG. 3A, the loading portion has an exterior configuration that is substantially similar to the configuration of the tapered interior sidewall located therein. That is, for example, the loading portion has a first substantially cylindrical exterior portion that extends a distance from a first end and then tapers from the first cylindrical portion along a substantially frusto-conical shape to the barrel. Those skilled in the art will understand and appreciate that the different exterior configurations of the body portion of the implanter 100 are not limited to the particular examples shown, as other shapes and configurations can also be utilized.

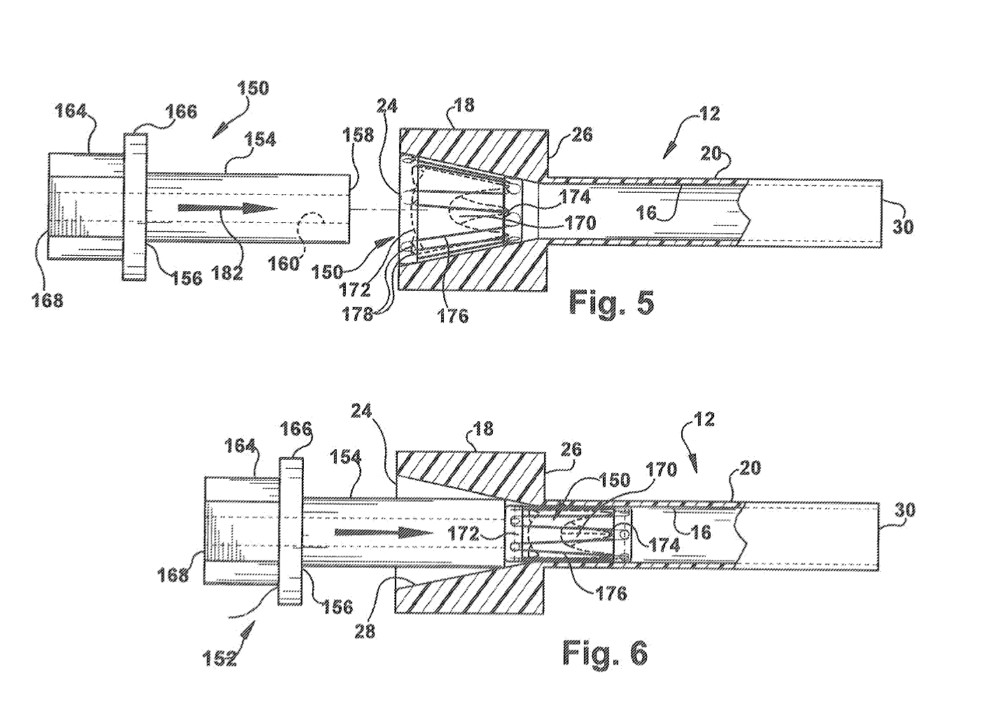

FIGS. 5 and 6 depict different parts of an example procedure that can be employed to load a prosthesis 150 into the barrel 20 of an implanter 10. For purposes of simplicity explanation (but not by way of limitation), the procedure is implemented using the implantation system 10 shown and described with respect to FIGS. 1 and 2. Accordingly, the same reference numbers in FIGS. 5 and 6 refer to parts and structure previously introduced with respect to FIGS. 1 and 2. Those skilled in the art will understand and appreciate that a similar procedure can be utilized to load a prosthesis into other configurations and arrangements of implantation systems that may be implemented.

In the example of FIGS. 5 and 6, one or more pusher members 152 can be utilized to help urge the prosthesis 150 into the loading portion 18 of the implanter body 12 and into the barrel 20 of the implanter 10. The pusher member 152 includes at least one elongated rod 154 that extends axially from a first end 156 and terminates in a second end 158. The second end 158 of the rod 154 can be substantially flat (e.g., substantially planar) or otherwise configured for engaging an end of the prosthesis. An aperture 160 may extend axially through the pusher member 152, such that one or more elongated articles can pass through the pusher member as it is used. The diameter of the rod 154 may be fixed along its length, such as having a size that is between the diameter of the opening 22 of the loading portion 18 and the inner diameter of the barrel 20 adjacent the end 26. For example, by dimensioning the diameter of the rod 154 to approximate the diameter of the barrel 20 and providing the elongated rod 154 with an axial length that is at least equal to or greater than the axial length of the loading portion 18, the rod can be inserted completely into the loading portion 14.

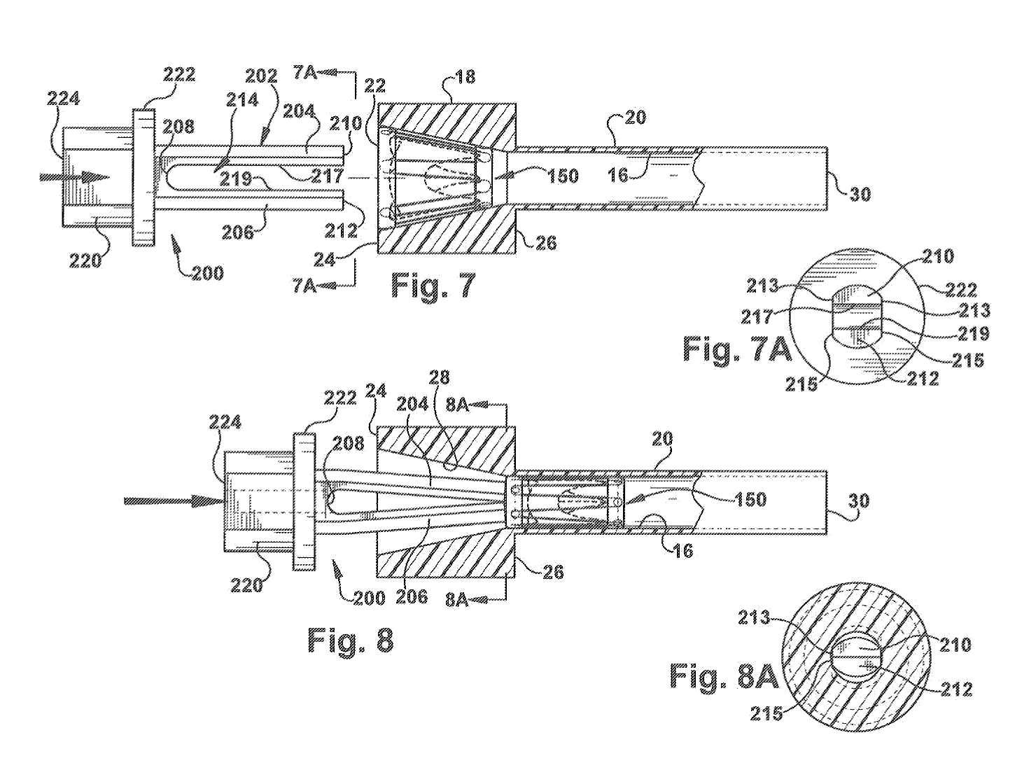

As an alternative, the rod 154 can be configured as including two (or more) spaced apart and opposing elongated members configured to provide a variable diameter. For example the variable diameter can decrease from a starting diameter by radially inwardly deflection of the two or more elongated members toward the central axis, such as in response to engaging the sidewall 28 during insertion into the loading portion 14 (see, e.g., FIGS. 7 and 8).

In the example of FIGS. 5 and 6, the pusher member 152 includes a second elongated rod 164 that extends axially from a spacer 166 to terminate in a distal end 168. The second rod 164 can be coaxial with the first rod 154, although it need not be coaxial (e.g., it might be transverse or oriented at other angles relative to the first rod). The second rod 164 also has diameter that may be substantially fixed along its length, and which is different from the diameter of the rod 154. For example, the diameter of the second rod 164 can be less than the diameter of the opening 22 and greater than the diameter of the first rod 154, such that the rod 164 can be inserted axially (at least partially) into the passage defined by the sidewall 28.

The spacer 166 extends radially outwardly from the pusher member 152 at an axial location that is between the first and second rods 154 and 164, respectively. The spacer 166 thus separates the rods 154 and 164. The spacer 166 can also extend radially beyond the exterior of each of the rods 154 and 164 with a cross-sectional diameter that is greater than the diameter of the opening 22. By configuring the spacer 166 to be diametrically larger than the opening 22, it provides a convenient handle for grasping the pusher member 152. The spacer 166 can also engage the end 24 of the loading portion 18 to inhibit insertion of the pusher member beyond some predetermined distance.

By way of example, assuming that the rod 164 has a greater cross-sectional diameter than the rod 154, the larger diameter rod 164 can be used to urge the prosthesis 150 into the loading portion 18 while the prosthesis itself has a greater diameter (e.g., in an expanded condition). After the prosthesis 150 has been inserted a first amount using the second rod 164, the user can flip the pusher member 152 (e.g., 180 degrees) so that the first, smaller diameter rod 154 is axially aligned with and adjacent the opening 22 of the loading portion 18. The user can employ the rod 154 to push the prosthesis 150 further through the loading portion 18 and at least partially into the lumen 16 of the barrel 20.

By way of further example, the procedure shown in FIGS. 5 and 6 is shown in the context of loading a heart valve prosthesis 150 into the body of the implanter 10. It will be understood that the procedure is not limited to use with a heart valve prosthesis as other types of implantable prostheses, such as annuloplasty rings, stents, supports and other devices, can be used.

The prosthesis 150 includes a valve 170 that is configured to provide for substantially unidirectional flow of blood through the valve. In the example of FIGS. 5 and 6, the valve 170 includes a plurality of leaflets that extend radially inward from the sidewall portion of the valve. The leaflets are moveable into and out of engagement with each other to coapt for providing unidirectional flow of blood through the valve 170. For different types of valves, there may be different numbers of leaflets or other moveable means (e.g., a ball, a flap or other structure) that provide for the desired unidirectional flow of blood through the valve. Additionally, the valve 170 can be a homograft or xenograft or, alternatively, the valve can be constructed of natural tissue, synthetic or a combination of natural and synthetic materials that are connected together to provide the valve. Those skilled in the art will understand and appreciate other types of valves that can be utilized, including those mentioned herein as well as others yet to be developed.

As one example the prosthesis 150 can be a natural tissue heart valve prosthesis that includes a valve 170 having an inflow end 172 and an outflow end 174 at axially opposed ends of the valve. The valve 170 is mounted within a support 176. For instance, a sidewall portion of the valve 170 extends between the ends 172 and 174 of the valve, and between corresponding ends of the support 176. For instance, the inflow end 172 of the valve 170 is positioned near an inflow end 172 of the support 176 and the outflow end 174 of the valve is positioned near an outflow end of the support. The outflow end 174 of the valve 170 can have a generally sinusoidal contour, as shown in FIG. 5, although the valve is not limited to such an outflow contour. For the example valve 170, the peaks of sinusoidal outflow end can be aligned generally with and attached to support junctures near the end of the support 176. The valve 170 can be connected within the support 176 via sutures or other known connecting means, for example. The prosthesis 150 may also include an outer sheath of a substantially biocompatible material (e.g., a natural or synthetic material). The outer sheath can cover at least a substantial amount of exposed portions of the support 66, such as including the ends 70 and 72, to mitigate contact between the blood and the support when the prosthesis is implanted.

In the example of FIGS. 5 and 6, the support 176 is configured to enable the valve to be compressed to a reduced cross-sectional dimension (diameter) and then expanded back to an expanded and functional condition. The support 176 can be self-expanding from its reduced cross-sectional dimension or it may be expandable by employing other means to expand the valve manually (e.g., balloon catheter or other radially expanding mechanism). The support 176 includes substantially axially extending support junctures that are configured as arcuate junctures that are biased so as to urge a pair of adjacent axially extending support features circumferentially apart.

The support 176 further can include includes one or more projections or spikes 178 that extend axially and radially outwardly from at least some of the respective end junctures of the support. While a pair of such spikes 178 is illustrated as associated with each end juncture, other number of spikes can be implemented, such as single spike or more than two spikes at some or all of the junctures.

According to one example, the support 176 can be formed a shape memory material, such as NITINOL. For example, the support 176 can be formed from a small cylindrical tube of the shape memory material, such as via a laser cutting (ablation) process in which the desired sinusoidal sidewall is cut from the tube. In this way, the support features, the interconnecting end junctures, and associated spikes 178 can be formed as a monolithic structure (e.g., integrally formed) having a desired shape and size. Additionally, ends of the spikes 178 can have tapered or sharpened tips to facilitate gripping surrounding tissue when implanted. For example, the spikes 178 can be formed by laser cutting from the same tube or, alternatively, they could be welded onto or otherwise attached to the support 66 at desired positions. The resulting structure can then be heated to its transformation temperature and forced to a desired cross-sectional dimension and configuration (its austenitic form. The support 176 can then be bent or deformed to a reduced cross-sectional dimension when in its low-temperature (martensitic) form to facilitate its mounting within a barrel 20 of the implanter 10, for example.

Those skilled in the art will appreciate various other materials that may be utilized for the support 176, including elastically deformable and inelastically deformable materials, such as metals, alloys and plastics or other polymers and combinations of materials. By elastically deformable, it is meant that the structure is capable of sustaining stress without permanent deformation, such that it tends to return substantially to its original shape or state when the applied stress is removed (e.g., self expanding from its reduced cross-section). By inelastically deformable, it is meant that the structure substantially retains its deformed shape after sustaining stress, such that it bends and stays bent until deformed to another (e.g., its original) shape or configuration. Additionally, if something is described herein as being deformable it may be either elastically deformable or inelastically deformable or exhibit different characteristics of one or both of such deformability.

By way of further example, the loading procedure can begin by selecting the appropriate prosthesis, which in the illustrated example is an expandable type natural tissue heart valve prosthesis 150, as described above. As described herein, however, the implantation system 10 is not limited to use with such a heart valve prosthesis. The initial alignment and insertion of the prosthesis 150 into the loading portion 18 can be implemented manually (e.g., by hand). Once the prosthesis 150 has been appropriately aligned and, optionally, inserted into the opening a small amount (e.g., about 2-5 mm), the pusher member 152 can be employed to urge the prosthesis 150 farther into the loading portion 18.

The pusher member 152 can urge the prosthesis in the direction of arrow 182 axially into the passage provided by the interior sidewall 28 of the loading portion 18. The engagement between the sidewall 28 of the loading portion 18 and the exterior of the prosthesis 150, as the prosthesis is urged axially into the loading portion, operates to compress the prosthesis 150 to a reduced cross sectional dimension, as shown in FIG. 5. For instance, the inflow end 172 the prosthesis remains in a substantially expanded condition, whereas portion of the prosthesis sidewall proximal the outflow end (located within the interior sidewall 28 of the loading portion 18) tapers along its length according to the dimensions and configuration of the interior sidewall 28 in which it is being inserted. The relative axial position at which the interior sidewall 28 begins to urge the prosthesis 150 to a reduced cross-section will vary according to, for example, the relative dimensions of the implanter body parts and the size of the prosthesis.

After the rod 164 has been inserted into the loading portion 18 such that it cannot be inserted further (e.g., the end 158 engages the sidewall 28 or the central spacer 166 engages the rim at the opening 22), the pusher member 152 can be flipped around to use the smaller diameter rod 154. For example, in FIG. 5, the rod 154 is axially aligned with the prosthesis 150 and body lumen 16. The end 158 of the pusher member 152 can, in turn, be urged into engagement with the adjacent end of the prosthesis 150 so as to insert the prosthesis into the loading portion 18 for loading the prosthesis farther into the barrel 20 of the implanter 10, such as shown in FIG. 6.

The rod 154 (having a smaller diameter than the rod 164) thus can be inserted axially into the loading portion 28 of the further than the rod 164. The distance that the rod 32 can be inserted will generally depend on the relative diameters of the rod and the sidewall 28. In the example of FIG. 6, the rod 154 is inserted approximately ¾ the length of the sidewall 28 of the tissue receiving portion when the end 158 engages the sidewall so as to inhibit further movement into the loading portion 18. It will be appreciated that the rod 154 and interior sidewall 28 of the loading portion 18 could be provided at different relative dimensions from those shown so as to provide for different depths of insertion. Additionally, more than two rods can be provided to allow for additional levels axial insertion. For smaller size barrels (having a diameter from about 7 mm to about 9 mm), the pusher can include one or more rods configured to have a variable diameter so that the pusher member 152 can be inserted axially at or adjacent to juncture between the barrel 20 and the loading portion 18.

FIGS. 7, 7A, 8 and 8A depict an example of another type of pusher member 200 that can be utilized to load a prosthesis or other implantable device into an implanter 10. For sake of simplicity of explanation, the implanter body 12 and prosthesis 150 will be described as being substantially the same as, as shown and described with respect to FIGS. 5 and 6. It will be appreciated, however, that the implanter body 12 and/or prosthesis can be the same or different from that shown and described with respect to FIGS. 5 and 6.

The pusher member 200 includes at least one elongated rod assembly 202 having two elongated rod members 204 and 206. The rod assembly 202 is not limited to only two rod members 204 and 206, as more than two rod members can be implemented (e.g., a substantially circumferential array of three, four or more axially extending rod members spaced apart from each other). In the example of FIGS. 7 and 8, each of the rod members 204 and 206 are joined at and extend axially from a first end 208 and terminate to define respective opposing second ends 210 and 212 of the pusher member. The second ends 210 and 212 of the rod members 204 and 206 are substantially flat (e.g., substantially coplanar) or otherwise configured for engaging an adjacent end of the prosthesis 150.

In the example of FIGS. 7 and 7A, the rod members 204 and 206 are coextensive and substantially parallel and spaced apart from each other by slot or notch 214 that extends continuously and axially from the end 208 to the open end between the ends 210 and 212. The first end 208 can operate as a hinge that permits the ends 210 and 212 of the rod members 204 and 206 to deflect radially inwardly relative to the central axis (and toward each other) to reduce the distance between the opposing side surfaces of the respective rods. Additionally, diametrically opposed side edges 213 and 215 of the respective rod members 204 and 206 can be spaced apart from each other a distance that approximates the diameter (e.g., reference number 24 in FIG. 2) of the barrel 20. While in the example of FIGS. 6 and 7 the rod assembly 202 is depicted as an integral structure (e.g., monolithic), the rod members 204 and 206 could be fixed relative to each other by one or more other structures (e.g., hinge, spring, rivot, etc.) that permits desired movement (e.g., radially inward deflection) of the rod members to a reduced cross-section.

For example, radial thickness of each of the rod members 204 and 206 at the ends 210 and 212, respectively, can be dimensioned so that when the rod members deflect toward and each other, the total reduced thickness can approximates the diameter of the barrel 20. In this way, the variable diameter of the rod assembly 202 can decrease from a starting diameter (FIGS. 7 and 7A) and decrease radially due to inward deflection of the elongated rod members 202 and 204 toward each other. The deflection of the rod members 204 and 206 toward each other thus results in opposing inner surfaces 217 and 219 moving from a spaced apart condition (FIG. 7A) to a second condition in which the opposing surfaces are closer or contacting each other (FIG. 8A). Such inward deflection can occur in response to the exterior surface of the rod members 202 and 204 engaging the interior sidewall 28 of the loading portion 18 during insertion into the loading portion. Alternatively, the inward deflection of the elongated rod members may be manually adjustable, such as by application of external force or by otherwise adjusting the distance between the surfaces 217 and 219.

The pusher member 200 can include another rod 220 that extends axially from a spacer 222, which is located intermediate the rod 220 and the variable rod assembly 202. The rod 220 extends from the spacer 222 and terminates in a second end 224. The rod 220 can be coaxial with the first rod assembly 200, although it need not be coaxial (e.g., it might be transverse or at other relative angular orientations). In the example, of FIGS. 7 and 8, the rod 220 also has diameter which may be substantially fixed along its length, which can be larger than the starting diameter of the rod assembly 202. Alternatively, the rod 220 can be configured to have a variable diameter similar to the rod assembly 200, but have different starting and ending diameters. In this way, the rod 220 can be used for an initial phase of inserting the prosthesis 150 into the loading portion 18 (e.g., similar to as shown and described in FIG. 5). The variable rod assembly 200 can be used to complete the insertion of the prosthesis into and through the loading portion 18 and for loading the prosthesis into the barrel 20 of the implanter 10, such as depicted in FIG. 8.

After the prosthesis has been loaded into the barrel 20, the prosthesis is ready for implantation. As discussed with respect to FIGS. 1-4, the implanter system can include an introducer apparatus attached at a distal end of the barrel. The introducer apparatus facilitates insertion and penetration of the barrel into a desired anatomical structure, such as into a heart, a blood vessel or other structure.

FIG. 9 depicts the barrel 20 of an implanter 10 being urged axially through an introducer apparatus. For purposes of simplicity of explanation, the introducer apparatus 40 and implanter 10 will be described by referring to parts and structure with reference numbers previously introduced with respect to FIGS. 1 and 2. It will be appreciated that the introducer apparatus 40 and implanter are not limited to the previously shown and described structures, as various different configurations and uses may be envisioned within the scope of the appended claims.

As shown in FIG. 9, the retaining structure 66 can rest in a groove (or in one or more slots or notches) 230 to maintain the structure 66 at a desired axial position along the exterior of the sidewall 50 of the introducer apparatus 40. The structure 66 can be fixed (e.g., by an adhesive or ultrasonic welding) to a portion of the sidewall 50 or it can hold its position due to frictional forces. The structure 66 can be formed of a plastic, metal, rubber (e.g., a rubber-band-like or O-ring structure) or other material that can be employed to apply radially inward force to the sidewall 50. Thus, the structure 66 can be applied to urge the jaw members in a closed condition (e.g., as shown in FIG. 1).

FIG. 9 further depicts the barrel 20 inserted through the passage of the apparatus 40 such that the respective jaw members 54 are in an open condition around the barrel. As mentioned above, the jaw members 54 can be urged into the open condition by inserting the barrel 20 or other object through the opening associated with the inner periphery of the flange 42 and axially through the passage defined by the sidewall 50 of the introducer apparatus 40. In the example of FIG. 9, the barrel 20 has been inserted within the passage such that a distal end 30 of the barrel protrudes beyond the end 60 of the distal end portion 52.

To facilitate insertion of the barrel 20 through the distal end portion 52 of the introducer apparatus 40, as shown in FIG. 9, the radially inward force being applied by the structure 66 can be removed from the sidewall 50, such as by cutting or removing the part of the structure 66. The removal can be made through a slit or slot 232 formed in the flange 42 or by providing some mechanism for otherwise breaking the radially inward force applied thereby. When the structure 66 is cut, for example, the jaw members 54 can be more easily urged into their open condition so that the barrel 20 can substantially freely move through the passage defined by the interior of the introducer apparatus 40.

The jaw members 54 thus can be urged into their open condition in response to an exterior surface of the barrel 20 engaging an interior portion of the sidewall 50, which engagement causes the jaw members 54 to deflect outwardly away from the central axis. The material employed for the jaw members 54 can exhibit resilience or some shape memory so that the jaw members return approximately to the closed condition after the barrel 20 has been withdrawn from the passage defined by the sidewall 50. Alternatively, the jaw members 54, depending on the material construction, might be inelastically deformable to remain in a generally open condition upon removal of the barrel 20.

In the example of FIG. 9, an exterior surface of the barrel 20 can also include indicia 236 that can be utilized to ascertain the length of the barrel that has been inserted through the introducer apparatus 40. For instance, a proximal end of the introducer apparatus 40 (e.g., a portion of the flange 42) can align with circumferentially extending indicia 236 to indicate a measurement as to how far the distal end 30 of the barrel 20 has been inserted through the introducer apparatus, such as corresponding to a distance beyond the flange 42. This can be used, for example, to position the end 30 of the barrel 20 accurately relative to anatomical structures within the patient, such as in an organ or other implantation site.

Additionally or alternatively, the introducer apparatus 40 can include another retaining structure 238 applied to the annular flange 42 to inhibit movement of the barrel 20 through the passage defined by the sidewall 50. In the example of FIG. 9, the retaining structure 238 is depicted as a ring (e.g., an O-ring) applied to and engaging an outer periphery of the flange 42. To facilitate holding the structure 238 to the flange 42, the outer periphery can be recessed or include a groove around the flange.

One or more slits (or notches) 232 can also extend through the flange 42 to provide an adjustable cross-sectional dimension for the flange. The one or more slits 232, for example, can extend radially at least from the outer periphery to the inner periphery of the flange, and may further extend along a proximal part of the sidewall 50, such as shown in FIG. 9. Thus, by applying the structure 238 to the outer periphery of the flange 42, the slits 232 can be urged toward a closed condition (FIG. 1) so that the inner periphery of the flange 42 exerts radially inward force along an exterior of the implanter barrel 20. This radially inward force (while the structure 238 is applied) inhibits axial movement of the introducer apparatus 40 relative to the barrel 20. As a result, manipulation and insertion of the combination of the barrel 20 and introducer apparatus 40 into anatomical structures can be facilitated, such as for implanting a prosthesis or other device. After the introducer apparatus 40 has been inserted so that the flange 42 engages the anatomic structure in which the implantation site resides, the structure 238 can be cut or removed from the flange (as shown in FIG. 9) to remove the radially inward force being applied. Thus, when the force being applied by the structure 238 has been removed, the barrel 20 can move substantially freely through the apparatus 40 to facilitate positioning the distal end 30 of the barrel 20 at or near the desired implantation site.

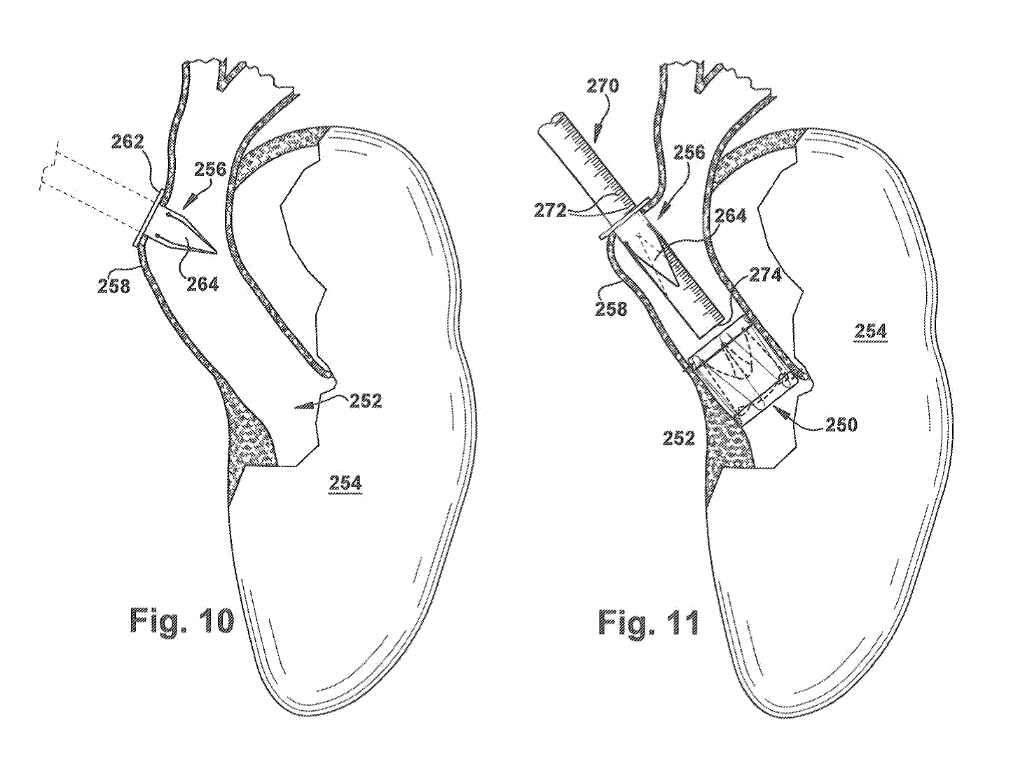

FIGS. 10 and 11 depict and example of a portion of a procedure that can be utilized for implanting a heart valve prosthesis 250 at an aortic annulus 252 of a patient's heart 254. In the example of FIGS. 10 and 11 it is assumed that at least portions of a defective valve or the entirely defective valve that is being replaced has been removed from the aortic position. Those skilled in the art will understand and appreciate that some heart valves might alternatively be implanted while the defective valve remains intact. Additionally, as described herein, different types of implantable articles might be implanted in a similar way to improve operation of a patient's defective valve.

In FIG. 10, an introducer apparatus 256 has been inserted into the aorta 258. As an example, an incision is made at a desired location in the aorta 258 of the patient's heart 254 and the introducer apparatus 256 is inserted into the incision, such that a flange 262 of the introducer apparatus engages the exterior wall of the aorta 258. A purse string (not shown) can be applied around the insertion to mitigate bleeding by tightening the aortic wall around the sidewall of the introducer apparatus 256. The introducer apparatus 256 can be inserted separately or in combination with the implanter 270, such as while the implanter is inserted partially into the introducer apparatus. Once the introducer apparatus 256 has been inserted, as shown in FIG. 10, a distal end portion 264 of the introducer apparatus 256 extends into the aorta 258 in the closed condition. The introducer apparatus 262 can be constructed according to any of the types described herein, such as shown and described herein FIGS. 1, 2 and 9. Those skilled in the art will understand and appreciate that differently sized and configurations of introducer apparatuses can be utilized, for example, depending upon the location in which such apparatuses are to be implanted and the dimensions and configuration of the implanter. Additionally, the introducer apparatus 256 can be inserted into other anatomical structures, including other blood vessels (e.g., pulmonary artery), to provide access to an intended implantation site.

In the example of FIG. 10, the introducer apparatus 256 is inserted into a position such that a generally direct (e.g., linear) path can be provided from the introducer apparatus to the desired implantation site (e.g., the aortic annulus 252) for implanting the prosthesis 250. As mentioned above, an O-ring or other means (not shown) can be applied to a sidewall of the introducer apparatus 254 to help hold the jaws closed during its insertion into the heart 254 as well as to inhibit passage of the barrel of the implanter through the distal end of the introducer. The path from the introducer apparatus 256 to the implantation site 252 can be substantially linear or it can provide a substantially curved or indirect path depending upon the type of implanter being utilized.

FIG. 11 depicts an example of the heart valve prosthesis 250 being implanted at the aortic annulus 252 by employing an implanter 270 in combination with the introducer apparatus 256. Those skilled in the art will understand and appreciate various types of implanters 270 that can be utilized in conjunction with an introducer apparatus based on the teachings contained herein. As described herein, indicia 272 along the sidewall of the implanter 270 may provide means for determining a measure of the distance that an end 274 of the implanter barrel has been inserted to within the aorta 258. Thus, the measurement information from the indicia 272 can facilitate discharging the prosthesis 250 from the implanter at a desired implantation site 252. The distance and location of the implantation site 252 can be ascertained by employing a number of techniques. For example, the end 274 of the implanter 270 can be positioned via ultrasonic or radiographic means, such as a cardiac 3-D echo performed before and/or during the implantation procedure.

It is to be appreciated that the prosthesis 250 may be implanted at the aortic annulus 252 during a conventional open chest procedure or during a closed chest procedure. Because the only incision is in the patient's aorta, the implantation can be performed during very short open chest surgery, for example, with reduced cardiopulmonary bypass when compared to many existing procedures. It is to be understood and appreciated that if the patient has a calcified aortic valve, the patient typically will be put on cardiopulmonary bypass to remove the defective valve or at least calcified portions thereof and to implant the prosthesis 250. A prosthesis 250 may still be implanted more effectively than many conventional approaches even when cardiopulmonary bypass is utilized.

In the example of FIG. 11, the heart valve prosthesis 250 is depicted as a valve that can be implanted without requiring sutures (e.g., referred to as a sutureless valve), although sutures can be used. It is to be understood and appreciated that various types of valves configurations of could be employed to provide the heart valve prosthesis 250. Additionally, the implanter 270 is not limited to implantation of a heart valve prosthesis, as other types of expandable prosthetic and other implantable devices can be implanted using an approach to similar to that of FIGS. 10 and 11.

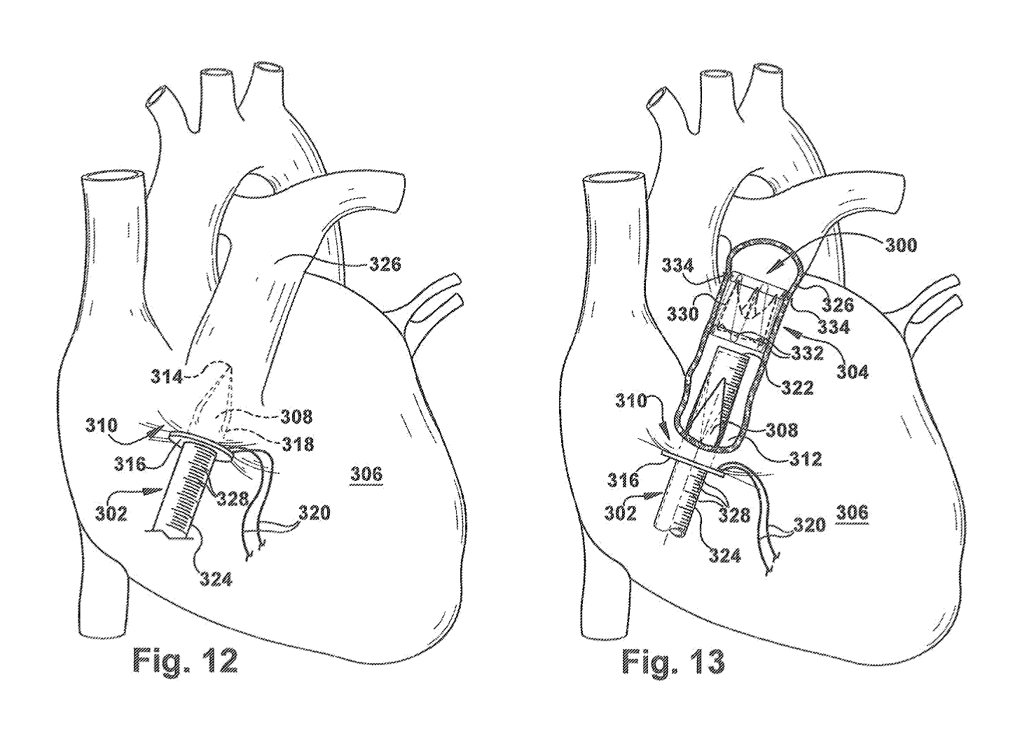

FIGS. 12 and 13 depict another example procedure that can be utilized for implanting a heart valve prosthesis 300 using an implanter 302. In the example of FIGS. 12 and 13, the prosthesis 300 is implanted at a pulmonic position 304 of a patient's heart 306. In the example of FIG. 12, an introducer apparatus 308 has been inserted into an anterior wall 310 of the right ventricle 312 of the heart 306, such that a distal end portion 314 of the introducer apparatus extends into the right ventricle of the heart. The insertion location into the heart 306 can vary to provide a substantially direct (e.g., linear) path to the implantation site at the pulmonic position 304. A flange portion 316 of the introducer apparatus 308 engages the exterior surface of the anterior wall 310 of the right ventricle 312. An intermediate portion 318 of the introducer apparatus 308 thus can be surrounded by the heart muscle 306 adjacent the anterior wall 310 of the heart. The engagement of the surrounding heart muscle 306 with the introducer apparatus 308 can be facilitated, for example, by applying a double purse string suture 320 generally around the insertion location, such as prior to making an incision through the heart 306 into the right ventricle 312.

The distal end portion 314 of the introducer apparatus 308 is in the closed condition during insertion of the introducer apparatus into the heart muscle 308. The closed condition of the distal end portion 314 can be facilitated by employing a ring or other structure to retain the closed condition, such as shown and described herein. The location in which the introducer apparatus 308 is inserted into the heart 306 generally will depend on the type of implanter and the location of the implantation site. In some cases, such as implantation of a valve into the heart muscle for an extra-anatomic procedure, the introducer apparatus 308 may be omitted.

In FIG. 13, a distal end 322 of the barrel 324 has been inserted through the introducer apparatus 308 that so that respective jaw members of the distal end portion 314 are urged apart to the open condition by the barrel 324 (see, e.g., FIG. 9) and thereby permit substantially free movement of the barrel through the introducer apparatus 308. By inserting the introducer apparatus 308 through the anterior wall 310 proximal the pulmonary artery 326, a substantially direct or generally linear implantation of the prosthesis 300 can be performed with little or no cardiopulmonary bypass. As mentioned above, however, when the patient's defective valve is calcified, cardiopulmonary bypass will typically be performed for removing the calcified valve portions prior to implantation. The removing of the calcified valve can be performed, for example, by employing a trocar or other tube inserted through the introducer apparatus 308 or during an open heart procedure prior to implanting the prosthesis 300.

Those skilled in the art will understand and appreciate other possible paths through the heart or associated arteries or veins that could be employed for positioning the distal end 322 of the barrel 324 to enable implantation of the prosthesis 300. The barrel 324 further can include indicia 328, such as corresponding to ruler markings, for providing an indication of measurement as to the distance which the end 322 of the barrel 324 has been inserted into the heart 306. Accordingly, the measurement from the indicia 328 enables a user to discharge the prosthesis 300 into the pulmonary artery 326 at the desired implantation site, namely at the pulmonic position 304. Once at the desired position, the prosthesis 300 can be discharged or ejected from the barrel 324 into the outflow of the right ventricle 312, as illustrated in FIG. 13 and further described with respect to operation of the implanter.

The prosthesis 300 can be substantially similar to that shown and described with respect to FIG. 7, although other types of valves can also be utilized. Additionally or alternatively other types of prostheses (e.g., annuloplasty rings, stents, supports and the like) or implantable devices can be implanted using the implanter 302. In the example of FIGS. 12 and 13, the prosthesis 300 can include an expandable type of support 330. For a sutureless type of implantation, the prosthesis 300 can also include spikes or protruding portions 332 for engaging surrounding tissue of the pulmonary artery 326 in its implanted position. The spikes or protruding portions 332 thus inhibit axial and/or angular movement of the implanted prosthesis 300. While the spikes or protruding portions 332 are depicted as extending generally arcuately from the respective end portions, it will be appreciated that suitable spikes or protruding portions can be located at any external location of the support 330. It will be appreciated, however, that one or more sutures 334 can also, or alternatively, be employed as a means to secure the valve relative to the pulmonary artery 326. For instance, sutures 334 can be applied externally to prosthesis 300 after its implantation, or sutures can be applied internally, such as through a passage defined by the barrel 324.

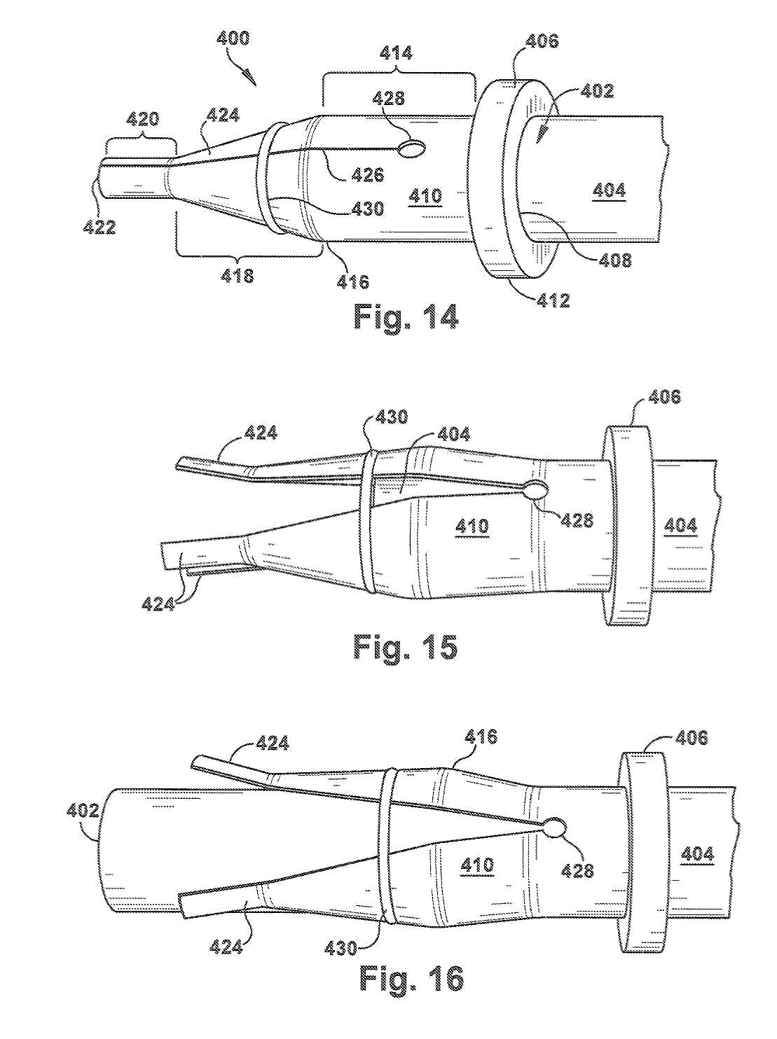

FIGS. 14, 15 and 16 depict another example of an introducer apparatus 400 that can be utilized for implanting a cardiac prosthesis. Like reference numbers are used in FIGS. 14-16 to identify corresponding parts of the introducer apparatus and the barrel. In the example of FIG. 14 the introducer apparatus 400 is attached adjacent a distal end 402 of a barrel 404. The introducer apparatus 400 includes a flange 406 extends radially outwardly from a proximal end of the introducer apparatus. While the flange 406 is illustrated as a complete annular flange, those skilled in the art will understand and appreciate that the flange 406 can be implemented in a variety of shapes (e.g., rectangular hexagonal, etc.) and that flange 406 need not extend in a complete annular structure. For example, the flange 406 can be implemented as substantially co-planner tabs, a rim, as well as a curved or c-shaped flange portion or as a thicker sidewall portion of the apparatus 40.

The flange 406 can serve dual purposes; namely, prevent insertion of the introducer apparatus 400 into tissue beyond the flange (e.g., the flange engages tissue or muscle) as well operate as an adapter that can be gripped by user to slide the introducer apparatus 400 backwardly along the barrel of the introducer during use.

In the example of FIGS. 14-16, the flange 406 includes an inner periphery 408 that defines an opening into an associated sleeve portion 410. While the inner periphery 408 and the sleeve portion 410 are demonstrated as having a generally circular cross-sectional shape in the examples of FIGS. 14-16, other shapes can be implemented, generally corresponding to the cross-sectional shape of the barrel 404.

The flange 406 also includes an outer peripheral edge 412 spaced apart from the inner periphery 408 by an associated intermediate portion thereof. The sleeve portion 410 has a cylindrical sidewall 414 that extends longitudinally between the flange 412 and an end thereof, indicated at 416. The sidewall 414 defines a first proximal portion that includes an interior diameter that is dimensioned about the same as (or slightly less than) the outer diameter of the barrel 404, such that the barrel can slide through the hollow passage extending therethrough.

An intermediate portion 418 of the introducer apparatus 400 extends between the end 416 of the first proximal portion 414 and a tip portion 420. The sidewall of the intermediate portion 418 thus tapers from the end 416 (e.g., at the dimension of the first proximal portion 414) a reduced cross-sectional dimension of the tip portion. In the example of FIGS. 14-16, the intermediate portion 418 thus has a frusto-conical configuration. The tip portion 420 in turn extends longitudinally from the intermediate portion to terminate in a distal end 422. For instance, the tip portion 420 can have as substantially constant diameter (e.g., less than about 1 cm) such as depicted in FIG. 14.

A central axis extends longitudinally through a center of the introducer apparatus 400. Similar to the other example, the distal end portion of the introducer apparatus can move between a closed condition (FIG. 14) and an open condition (FIGS. 15 and 16). The adjustable opening thus provides means for moving from at least a closed condition (FIG. 14) to an open condition (FIG. 9), such as to permit substantially free movement of an article, such as an implanter barrel or trocar, axially through a passage defined by an interior of the introducer apparatus 400.

As an example, the adjustable opening can include two or more jaw members 424 that are movable in a generally radially direction relative to the central axis between the open and closed conditions. In the example of FIGS. 14-16, the adjustable opening includes a plurality of three jaw members 424. Longitudinally extending side edges of adjacent jaw members 424 define longitudinally extending slits 426. The longitudinally extending slits 426 extend continuously through the sidewall of the introducer apparatus 400 from a first position 428, which is located in the sidewall 414 of the sleeve portion 410, through the intermediate portion 418, through the tip portion 420 and intersect through the distal end 422, such as to form separatable jaw members 424. In the example of FIGS. 14-16, the first position for each of the slits 426 is located axially at about the midpoint (e.g., one-half way) of the sidewall 414 of the sleeve portion 410.

An aperture further may extend through the sidewall 414 of the sleeve portion 410 at the first position 428 of each of the respective slits 426. The respective apertures operate as hinges to facilitate the generally radially outer movement of the respective jaw members 424 from the closed condition to the open condition. Other structure, such as hinges, can also be employed to facilitate movement of the jaw members 424 between the open and closed conditions. Additionally, one of the slits 426 might extend completely through the sidewall 50 as well as through the flange 42 to accommodate differently sized elongate members within the passage. Thus, the sidewall can be generally cylindrical or the sidewall can be generally C-shaped. Those skilled in the art will appreciate that the sleeve portion 410, the intermediate portion 418 and tip portion 420 may have other shapes than as shown and described herein.

The introducer apparatus 400 can be formed of a variety of materials including metals, alloys polymers and/or composites, although it should be made of a material that is considered biocompatible or that can be made sufficiently biocompatible for at least temporary insertion into a desired tissue of a patient. Additionally, the flange 406 and portions 410, 418 and 420 can be formed of the same or different materials. When formed of the same material, such as a plastic or thermoplastic material, the entire introducer apparatus 400 can be injection molded from a common material. Different materials can also be utilized for the various parts of the implanter 10.

The sidewall 50 of the end portions 410, 418 and 420 can have a thickness that can be selected according to the material utilized for the sleeve to enable desired movement of the distal end portion between the opened and closed conditions thereof. For instance, the end portions 410, 418 and 420, or at least the jaw members 424 thereof, can be formed of a flexible (e.g., pliant) material to facilitate movement of the jaw members from the closed to open condition, such as upon insertion of an elongate member therethrough. No amount of resilience or memory of the jaw members or memory of the material is required for implementing the introducer apparatus 400.

Additionally, the introducer apparatus 400 can include means, such as an annular structure (e.g., a flexible ring or rubber band) 430, to bias the jaws 424 toward the closed condition (FIG. 14). In FIGS. 14-16, the structure 430 is depicted as a ring mounted around an exterior of the intermediate portion 418 of the introducer apparatus 400. The annular structure 430 can apply radially inward force on the jaw members 424 at a position of the distal portion of the introducer apparatus having a smaller diameter than the barrel. For instance, the structure 430 can be positioned around jaw members 424 at a location that is axially spaced apart from the end 416 of the sleeve portion 410 in the direction of the tip. As a result, the annular structure can inhibit relative movement of the barrel through the introducer apparatus 400 until pushed with sufficient axial force while permitting movement of the barrel within the sleeve portion 410. Those skilled in the art will understand and appreciate other shapes (C-shaped or U-shaped) and configurations of structures that can be utilized implement the functions described herein as being performed by the structure 430.

In the example of FIG. 14, the barrel 404 is located within the apparatus 400 such that a distal end is within the sidewall 414 of the sleeve portion 410. Thus, the jaw members 424 remain in the closed condition. This closed condition with the barrel located within the sleeve portion 410 provides an implanter that can facilitate its insertion of introducer apparatus 400 into patient tissue (e.g., through an opening formed in such tissue). In FIG. 15, the end of the barrel 404 has been urged axially beyond the end 416 of the sleeve portion 410 such that the barrel engages an interior sidewall of the intermediate portion and thereby urges the jaws to move radially outwardly. In FIG. 16, the distal end of the barrel 404 has been urged axially through and beyond the distal end 422 of the introducer apparatus. When the barrel advances through the sleeve sufficiently to be within the patient's tissue, the introducer apparatus may be pulled out of such tissue to facilitate positioning of the distal end of the barrel at a desired implantation site. In this way, the jaw members 424 will not interfere with the surrounding anatomical feature near the implantation site.

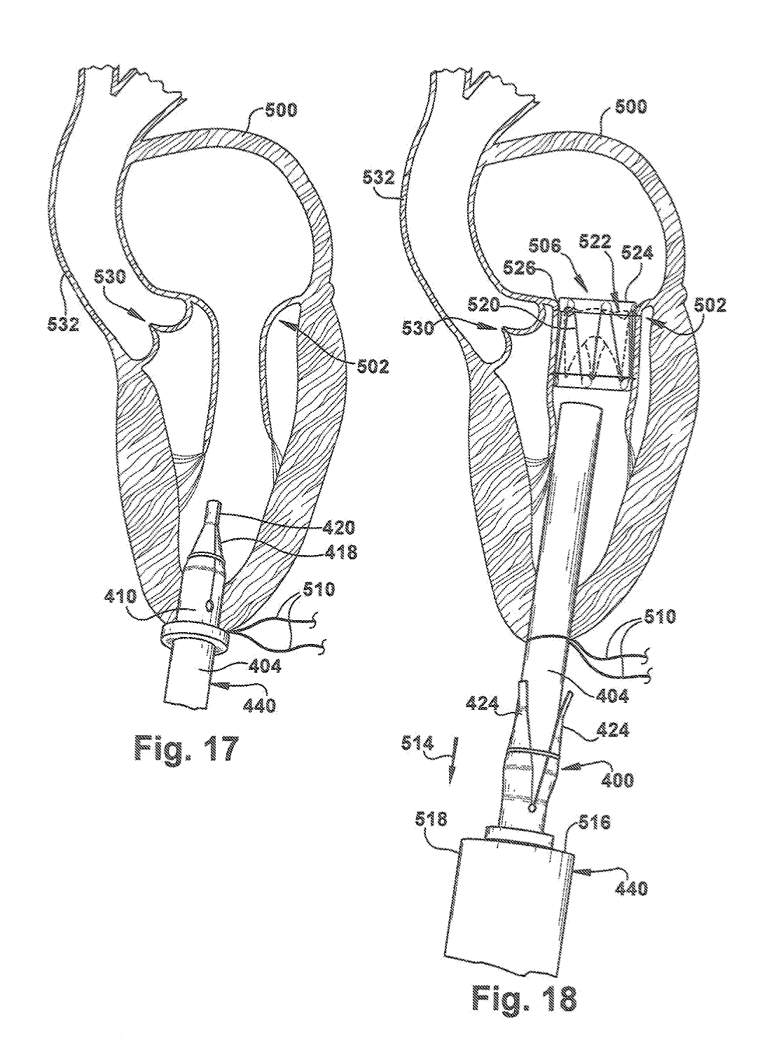

FIGS. 17 and 18 depict an example of procedure that can be used for implanting a cardiac prosthesis (e.g., a heart valve prosthesis, a stent, or other device) at desired implantation site in a patient's heart 500. In the example of FIGS. 17 and 18, the procedure is illustrated as being performed with the introducer apparatus 400 shown and described with respect to FIGS. 14-16. Accordingly, like reference numbers refer to part of the introducer apparatus 400 introduced with respect to FIGS. 14-16.

Additionally, in FIGS. 17 and 18, the procedure is shown and described with respect to implanting a prosthesis (e.g., a heart valve prosthesis) at the mitral annulus 502. It will be understood that the procedures shown and described herein, however, are not limited to implanting valves at illustrated position, but can be utilized to implant the valve at other positions (e.g., the aortic position, pulmonic position) as well as through openings formed in other places in the heart muscle (e.g., through a ventricular wall or atrial wall) or through major vessels (e.g., through the pulmonary artery or aorta). Moreover, while the implanter shown in the examples has a straight (e.g., linear barrel) in other embodiments, the barrel can be curved or inelastically deformable (e.g., bendable).

Turning to FIG. 17, the procedure begins after the appropriately sized and configured cardiac prosthesis 506 has been inserted into the barrel 404 of an implanter 440, such as shown and described herein. For example, the prosthesis 506 can be a heart valve prosthesis that has been reduced in its cross-sectional dimension and inserted in the barrel, such that upon being discharged it can return to its original size and configuration. This can be done automatically (e.g., the prosthesis can be self-expanding) or it may be assisted manually.

In the example of FIGS. 17 and 18, cardiopulmonary bypass is not required, as blood loss can be mitigated through application of a purse string suture 510 to the heart at the location through which the implanter is to be inserted (e.g., at the apex for implanting at the mitral or aortic positions). For example, after the patient's chest has been opened, a small incision or puncture can be made at the center of the purse string 510 that is applied around the location through which the introducer apparatus 404 and implanter 440 are to be inserted. The surgeon can then insert the tip portion 420, the intermediate portion 418 and at least a portion of the sleeve portion 410 through the opening in at the apex. The flange 406 is configured to prevent insertion of the introducer apparatus 400 into the heart or other area through which the prosthesis is being implanted. The purse string suture 510 can be tightened around the sidewall 410 of the introducer apparatus to mitigate bleeding. Consequently, cardiopulmonary bypass is not required. However, it is to be understood that in certain situations, such as when implanting the prosthesis at the aortic position, some bypass may be necessary, although usually for a much shorter period of time than with conventional procedures.

Once the introducer apparatus has been inserted into the heart, such as shown in FIG. 17, the surgeon can urge the distal end of the barrel to a location that is adjacent the implantation site. However, prior to discharging the prosthesis 506 the introducer apparatus 400 can be urged proximally and out of the heart (or other tissue) so as to mitigate interference by the jaw members 424. For instance, as shown in FIG. 18, a surgeon can pull the introducer apparatus 400 backward (in the direction of arrow 514) such that the flange engages the distal end 516 of the loading portion 518 of the implanter 440. The withdrawal of the introducer apparatus 400 can be performed concurrently with or before the barrel 404 is advanced toward the implantation site 502. The purse string sutures 510 can maintain desired closure of the heart (or other surrounding tissue) 500 around the barrel to mitigate bleeding.

After the distal end of the barrel 404, which can be flexible or bendable as described herein, has been inserted through the apex of the heart to the implantation position, the heart valve prosthesis 506 located in the barrel can be discharged. For instance, one hand of the surgeon can be used to advance the implanter toward the implantation site and an index finger (or other finger) on the other hand can be used to directly control guide the prosthesis to the desired implantation position. A plunger (or other pusher member) positioned for axial movement within the barrel 404 can be moved toward the distal end of the barrel and into engagement with the prosthesis 506 to discharge the prosthesis. As mentioned above, the heart valve prosthesis 506 can be configured to self-expand toward its fully expanded condition when it is discharged from the barrel 404.

In the example of FIG. 18, the prosthesis includes a support 520 and valve 522 are configured as shown and described in FIG. 18. That is, the support 520 includes a cage-like frame of axially extending resilient support features interconnected by biasing elements at axial ends thereof. The biasing elements can urge the support in a radially outward direction, such that it is self-expanding. It will be understood and appreciated that the implanter 440 can also be utilized to implant a non-expanding type of cardiac prosthesis or one that is already in its fully expanded condition within the barrel 404.

As shown in the example of FIG. 18, an inflow end 524 of the prosthesis 506 is annularized with respect to the mitral annulus 502 or other surrounding tissue (depending on the implantation site). An outflow portion 308 of the prosthesis 300 extends axially into the ventricle. Similar to as mentioned above (e.g., FIG. 11), a plurality of spikes 526 may extend laterally from the inflow end of the stent portion of the prosthesis 506 to engage the annulus 502 to help maintain a desired axial orientation of the valvular prosthesis. One or more sutures may also be applied.

While the example procedure of FIGS. 17 and 18 has been shown and described for implanting a prosthesis 506 at the mitral position 502 through the apex using the implanter 440, the same implanter and introducer apparatus 400 can be utilized to implant a valve at the aortic position 530 transapically. The result would be a cardiac heart valve prosthesis implanted at the aortic position similar to the result in FIG. 11 to provide for substantially unidirectional flow of blood from the ventricle into the aorta 532 (e.g., the valve would inverted compared to the valve in FIG. 18).

Moreover, while the foregoing examples demonstrate implanting a heart valve prosthesis in the heart of a patient, those skilled in the art will understand and appreciate that the introducer/implanter combination can be utilized to implant other types of implantable cardiac articles, such as annuloplasty rings, stents, as well as other devices. Additionally, the barrel of an implanter can be utilized as a trocar for performing surgical operations within the heart as may be required to facilitate adjustments of a prosthesis that has been discharged from an implanter or for adjusting the position of the heart valve prosthesis or other implanted device after its implantation. Additionally, those skilled in the art will understand and appreciate that the introducer apparatus and an implanter of sorts can be utilized for implanting other types of prosthesis and implantable devices associated with other anatomical structures or organs of the patient.

What has been described above includes examples or embodiments. It is, of course, not possible to describe every conceivable combination of components or methodologies for purposes of describing these examples or embodiments, but one of ordinary skill in the art will recognize that many further combinations and permutations are possible. Accordingly, the invention is intended to embrace all such alterations, modifications and variations that fall within the scope of the appended claims.

What is claimed is:

1. A method of implanting a cardiac prosthesis, comprising:
placing a cardiac prosthesis into an elongated barrel of an implanter, the barrel comprising an open discharge end;
attaching an introducer apparatus onto the open discharge end of the barrel, the introducer apparatus comprising a sidewall portion that extends from a first end portion and terminates in a distal end portion that gradually tapers to cross-sectional dimension that is less than cross-sectional dimension at the first end portion, the introducer apparatus being configured to permit movement of the barrel, including the discharge end, axially through and beyond the distal end portion of the introducer apparatus;
creating an opening in tissue that provides a path from the opening to an implantation site;
inserting the introducer apparatus into the opening;
moving the barrel axially through the introducer apparatus to position the discharge end of the barrel near the implantation site; and
discharging the prosthesis from the barrel at the implantation site.

2. The method of claim 1, wherein the introducer apparatus further comprises at least two jaw members, the at least two jaw members being urged radially outwardly relative to a central axis that extends through the introducer apparatus in response to movement of the barrel axially through the introducer apparatus.

3. The method of claim 2, further comprising biasing the jaw members toward a closed condition so as to resist the axial movement of the barrel through the introducer apparatus.

4. The method of claim 3, wherein the biasing further comprises providing an elastic structure that surrounds the jaw members along a tapering portion of the introducer apparatus that has a cross-sectional dimension that is less than an outer diameter of the barrel.

5. The method of claim 2, wherein the introducer apparatus comprises:
a sleeve portion having a sidewall portion dimension and configured to receive the barrel therein;
an intermediation portion extending from a distal end of the sleeve portion and tapering gradually to a reduced cross-sectional dimension; and
a tip portion that extends longitudinally from the intermediate portion to terminate in a distal tip that is spaced apart from a distal end of the intermediate portion,
wherein the at least two jaw members are formed from at least two axially extending slits through the sidewall of the tip portion, the intermediate portion and at least part of the sleeve portion, such that the movement of barrel through introducer apparatus causes corresponding parts of the at least two jaw members to move radially outwardly.

6. The method of claim 5, further comprising elastically resisting the radially outward movement of the jaw members by an elastic ring-like structure around at least part of the intermediate portion.

7. The method of claim 5, wherein the tip portion has a substantially constant cross-sectional dimension along its length extending between the intermediate portion and the distal end thereof, such that insertion of the introducer apparatus into the opening is facilitated.

8. The method of claim 5, wherein the tip portion, the intermediate portion and the sleeve portion comprise a monolithic integral structure of flexible material.

9. The method of claim 5, wherein the introducer apparatus further comprises a flange extending radially outwardly from a proximal end of the sleeve portion introducer apparatus, wherein during the inserting the flange engages tissue surrounding the opening so as to prevent further insertion of the introducer apparatus beyond the opening.

10. The method of claim 1, wherein prior to creating the opening, the method comprises placing a purse-string suture around the site where the opening is to be formed; and tightening the purse string suture around the introducer apparatus to mitigate bleeding.

11. The method of claim 1, wherein prior to advancing the barrel to the implantation site, the method further comprises pulling the introducer apparatus backward along the barrel such that it no longer resides within the opening in tissue.

12. The method of claim 1, wherein prior to discharging the prosthesis from the barrel, the method further comprises pulling the introducer apparatus backward along the barrel such that it no longer resides within the opening in tissue.

13. An implantation system comprising:
an elongated barrel extending longitudinally from a proximal portion to terminate in an open discharge end, a body lumen extending through the barrel to provide for fluid communication through the barrel;
a plunger configured for discharging a prosthesis from the discharge end of the barrel; and
an introducer apparatus that is attachable over the discharge end of the barrel, the introducer apparatus including a sleeve portion that extends from a first end and a distal portion that tapers gradually from the sleeve portion and terminates in a distal end thereof, the introducer apparatus being configured to permit movement of the barrel, including the discharge end, axially through and beyond the distal end of the introducer apparatus.

14. The implantation system of claim 13, further comprising at least two jaw members generally radially moveable relative to a central axis that extends through the distal portion of the introducer apparatus at least part of the sleeve portion of the introducer apparatus, the at least two jaw members being moveable relative to the central axis and to each other between the substantially closed condition and the open condition.

15. The implantation system of claim 14, further comprising means for biasing the jaw members toward the closed condition by applying radially inward force on part of the distal portion of the introducer apparatus that has an inner diameter that is less than the barrel.

16. The implantation system of claim 14, wherein the introducer apparatus comprises:
a sleeve portion having a sidewall portion dimension and configured to receive the barrel therein;
an intermediation portion extending from a distal end of the sleeve portion and tapering gradually to a reduced cross-sectional dimension; and
a tip portion that extends longitudinally from the intermediate portion to terminate in a distal tip that is spaced apart from the intermediate portion,
wherein the at least two jaw members are formed from at least two axially extending slits through the sidewall of the tip portion, the intermediate portion and at least part of the sleeve portion.

17. The implantation system of claim 16, wherein the tip portion has a substantially constant cross-sectional dimension along its length extending between the intermediate portion and the distal end of the introducer apparatus, such that insertion of the introducer apparatus into tissue is facilitated

18. A method of using the implantation system of claim 13, the method comprising:
placing a cardiac prosthesis into the barrel of the implanter;
attaching the introducer apparatus onto the discharge end of the barrel such that the barrel is inserted partially into the sleeve portion;
inserting the introducer apparatus into an opening in tissue that provides a path from the opening to the implantation site;
moving the barrel axially through the introducer apparatus to position the discharge end of the barrel at the implantation site that is spaced from the opening; and
discharging the prosthesis from the barrel at the implantation site.

19. The method of claim 18, wherein prior to advancing the barrel to the implantation site, the method further comprises pulling the introducer apparatus backward along the barrel such that it no longer resides within the opening in tissue.

20. The method of claim 18, wherein prior to discharging the prosthesis from the barrel, the method further comprises pulling the introducer apparatus backward along the barrel such that it no longer resides within the opening in tissue.
